(19) United States
(12) Patent Application Publication
(10) Pub. No.: US 2021/0347754 A1
(43) Pub. Date: Nov. 11, 2021

(54) Glycolide Production From Methyl Polyglycolate

(71) Applicant: Pujing Chemical Industry Co., Ltd., Shanghai (CN)

(72) Inventor: Wei LIU, Shanghai (CN)

(21) Appl. No.: 17/289,421

(22) PCT Filed: Oct. 29, 2018
(86) PCT No.: PCT/CN2018/112468

§ 371 Date: Apr. 28, 2021

(51) Int. Cl. C07D 319/12; C08G 63/08; C08J 11/24; C08J 11/26

(52) U.S. Cl. CPC C07D 319/12; C08G 63/08; C08J 11/24; C08J 2367/04; C08G 2115/00; C08G 2230/00; C08J 11/26

(57) ABSTRACT

The invention relates to a process of preparing a glycolide product from methyl polyglycolate or its product. The process comprises depolymerizing the methyl polyglycolate or its product in the presence of a depolymerization agent to make a depolymerized product; repolymerizing the depolymerized product to make a glycolic acid oligomer; and pyrolyzing the repolymerized mixture to make a glycolide product. A rare earth metal catalyst may be used facilitate the depolymerization reaction. The glycolide pyrolysis conversion rate may be greater than 90%. Also provided are related glycolide product and the glycolic acid oligomer. The glycolic acid oligomer may have a weight average molecular weight of 4,000-80,000.

### FIELD OF THE INVENTION

The present invention relates to glycolide production from a methyl polyglycolate or methyl polyglycolate product.

### BACKGROUND OF THE INVENTION

Methyl polyglycolate has good biocompatibility, excellent gas barrier properties, excellent mechanical properties, and excellent biodegradability. It is a polymer material with great potential and green environmental protection, which is in line with the development of today's society. The demand direction is towards wide use in biomedical devices, shale gas mining, packaging materials and other fields.

At present, all commercially available high molecular weight methyl polyglycolate products are prepared by ring-opening polymerization of glycolide. Due to thermal degradation and biodegradability of methyl polyglycolate, larger quantities of methyl polyglycolate waste products have been produced from, for example, scraps when materials are trimmed, or molded and disposable packaging materials and supplies that have been used or degraded in various ways are produced in manufacturing, processing, and uses of methyl polyglycolate. Although methyl polyglycolate can be eventually naturally degraded into water and carbon dioxide, improper handling will not only cause temporary environmental problems, but also waste resources, when these wastes are too concentrated for natural degradation. Low resource utilization and the loss of the polymer during the degradation process contribute to the current high price of methyl polyglycolate product.

Although many patents have reported the preparation of methyl polyglycolate by glycolide, there have been few reports on the recycling of methyl polyglycolate product. This is due mainly to the varying qualities, and varying degradation degrees of methyl polyglycolate which is hard to recover and recycle. Therefore, there is still a need for a stable, repeatable, effective, and low-cost industrial process for recycling various recovered methyl polyglycolate and methyl polyglycolate product of different qualities. Chinese patent CN101851227B discloses preparation of glycolide by catalytic pyrolysis of polyglycolic acid as a raw material under vacuum at 150-250° C., and the crude product of the obtained glycolide has a low purity so that it needs to undergo three recrystallizations, resulting in a glycolide yield as low as 53-60 wt %, and the utilization rate of raw material is very low. Therefore, this method does not achieve efficient recovery of polyhydroxyethanol raw materials to prepare high-purity glycolide.

Chinese patent application CN103781833A reports a method for recovering glycolide from polyglycolide. Specifically, the polymer in a molten form is first combined with a hydrolysis medium, and begins to be hydrolyzed to a polyglycolic acid oligomer, and then the oligomer is cyclized and depolymerized to form glycolide. The method is suitable for polyglycolide having a number average molecular weight of 134 to 10,000 g/mol, and is not suitable for high molecular weight polyglycolide. In addition, the residual hydrolysis medium has a great influence on the quality and purity of the glycolide product.

The above methods cannot meet the need for high-efficiency utilization of methyl polyglycolate and its products from a wide variety of sources, of different qualities and different degrees of degradation to obtain high-purity glycolide products. Therefore, efficient and convenient recovery of high-quality glycolide from methyl polyglycolate and methyl polyglycolate product is a problem that prior researchers have not overcome.

### SUMMARY OF THE INVENTION

The present invention relates to a process for producing glycolide from methyl polyglycolate.

A process of preparing a glycolide product from a methyl polyglycolate or a methyl polyglycolate product is provided. The process comprises (a) depolymerizing a methyl polyglycolate or methyl polyglycolate product in the presence of a depolymerization agent, whereby a depolymerized product is generated, wherein the depolymerized product comprises glycolate, glycolate polymer or glycolate prepolymer; (b) repolymerizing the depolymerized product, whereby a repolymerized mixture is generated, wherein the repolymerized mixture comprises a glycolic acid oligomer; and (c) pyrolyzing the repolymerized mixture, whereby a glycolide product is prepared.

The methyl polyglycolate or the methyl polyglycolate product may have a weight average molecular weight of 1,000-1,000,000.

The depolymerization agent may comprise one or more hydroxyl groups and comprises an alkyl alcohol, an alkyl alkyd or an alkyl alkanoate. The alkyl alcohol may comprise one hydroxyl group, preferably methanol. The alkyl alkyd may comprise one or more hydroxyl groups and one or more carboxyl groups at a number ratio of the hydroxyl groups to the carboxyl groups in a range from 0.1:1 to 10:1, preferably 1:1, more preferably glycolic acid. The alkyl alkanoate may comprise one or more hydroxyl groups and one or more ester groups at a number ratio of the hydroxyl groups to the ester groups in a range from 0.1:1 to 10:1, preferably a ratio of 1:1, more preferably methyl glycolate.

The depolymerization step (a) may be carried out at 25-260° C., preferably 30-190° C., more preferably 40-120° C., for 0.5-48 h, preferably 1-36 h, more preferably 2-24 h.

The process may further comprise applying microwave irradiation to control temperature in the depolymerization step (a) so that depolymerization is accelerated.

The depolymerization step (a) may be carried out in the presence of a rare earth metal catalyst. The rare earth metal catalyst may be a metal oxide, a rare earth metal inorganic salt or a rare earth metal complex comprising lanthanum (La), cerium (Ce), praseodymium (Pr), neodynium (Nd), promethium (Pm), strontium (Sr), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), scandium (Sc), or a combination thereof. The rare earth metal catalyst may be present in an amount of 0.0001-5 wt %, 0.0006-4 wt %, more preferably 0.001-2 wt %, based on the weight of the methyl polyglycolate or the methyl polyglycolate product.

The repolymerization step (b) may be carried out in the presence of a repolymerization catalyst. The repolymerization catalyst may be selected from the group consisting of zinc-based compounds, tin-based compounds, lanthanide-based compounds, and titanium-based compounds.

The repolymerization step (b) may be carried out at 160-250° C. and under an absolute pressure of 0.05-50.00 kPa.

The glycolic acid oligomer may have a weight average molecular weight of 4,000-80,000 and a free acid content of less than 2 wt %.

The pyrolysis step (c) may be carried out at 230-280° C. and an absolute pressure of 0.05-30.00 kPa.

The pyrolysis step (c) may be carried out in the presence of a viscosity reducer. The viscosity reducer may be present in an amount of 5-500 wt %, preferably 10-100%, more preferably 15-60, based on the weight of the methyl polyglycolic acid or the methyl polyglycolate product. The viscosity reducer may have a boiling point greater than 330° C. The process may further comprise distilling the glycolide product without the viscosity reducer.

The viscosity reducer may be a polyether polyol, preferably a polyethylene glycol, polypropylene glycol or polybutylene glycol, more preferably a polyethylene glycol having a weight average molecular weight between 1500 and 20,000, most preferably a polyethylene glycol having a weight average molecular weight of between 1,500 and 8,000.

The viscosity reducer may be a hydrocarbon mixture, preferably a mixture having a weight average molecular weight of less than 25,000, more preferably a hydrocarbon mixture having a weight average molecular weight of 1000-15,000, most preferably 1500-8000, and may be present in an amount of 5-500 wt %, preferably 10-100 wt %, more preferably 15-60 wt %, based on the weight of the methyl polyglycolate or the methyl polyglycolate product.

The pyrolysis step (c) may have a pyrolysis conversion ratio of more than 90% of the glycolic acid oligomer and generates an incompletely converted glycolide acid oligomer.

The process may further comprise discharging the incompletely converted glycolic acid oligomer as a residue at 230° C. with a dynamic viscosity less than 1 Pa·s.

A glycolide product is provided. The glycolide product is prepared from a methyl polyglycolate or a methyl polyglycolate product according to a process comprising: (a) depolymerizing a methyl polyglycolate or methyl polyglycolate product in the presence of a depolymerization agent, whereby a depolymerized product is generated, wherein the depolymerized product comprises glycolate, glycolate polymer or glycolate prepolymer; (b) repolymerizing the depolymerized product, whereby a repolymerized mixture is generated, wherein the repolymerized mixture comprises a glycolic acid oligomer; and (c) pyrolyzing the repolymerized mixture, whereby a glycolide product is prepared. The glycolide product may have a free acid content of less than 2%.

A glycolic acid oligomer is provided. The glycolic acid oligomer is prepared from a methyl polyglycolate or a methyl polyglycolate product according to a process comprising: (a) depolymerizing a methyl polyglycolate or methyl polyglycolate product in the presence of a depolymerization agent, whereby a depolymerized product is generated, wherein the depolymerized product comprises glycolate, glycolate polymer or glycolate prepolymer; and (b) repolymerizing the depolymerized product, whereby a glycolic acid oligomer is prepared. The glycolic acid oligomer may have a weight average molecular weight of 4,000-80,000. The glycolic acid oligomer may have a free acid content of less than 2 wt %.

### DETAILED DESCRIPTION OF THE INVENTION

The present invention relates to a process of producing glycolide from methyl polyglycolate or a product made from methyl polyglycolate. The inventors have surprisingly discovered that a glycolic acid oligomer having a weight average molecular weight of 4,000-80,000 may be obtained by depolymerizing and repolymerizing methyl polyglycolate and may be used to produce high-purity glycolide by pyrolysis. After depolymerization, repolymerization and pyrolysis, the glycolide products are produced steadily and solve the problem of resource waste and waste disposal in the production, manufacture and use of methyl polyglycolate.

Recovered methyl polyglycolate (including but not limited to waste in the production of methyl polyglycolate, trim in the processing of methyl polyglycolate, and polyglycolate products that have begun to degrade after use) have a wide molecular weight range. The weight average molecular weight range is approximately 1,000-1,000,000. The direct pyrolysis of these raw materials of different qualities is difficult to do while also ensuring a constant product quality. Methyl polyglycolate having very large molecular weight and very high melt viscosity are therefore difficult to process in the melt. It is difficult to reach a pyrolysis conversion rate greater than 90 wt % with very high molecular weight methyl polyglycolates. After pyrolysis, the remaining unconverted raw materials are discharged as a residue. When the molecular weight of the starting material for the pyrolysis is very high, more residues are produced due to slagging and the efficiency of the pyrolysis is very low due to the high viscosity, as mentioned above.

The terms “depolymerized product” comprises glycolate, a glycolate polymer, a glycolate prepolymer or a combination thereof.

The terms “glycolic acid oligomer” and “glycolate prepolymer” are used herein interchangeably and refer to a polymer comprising glycolic acid or glyoclate repeating units and having a weight average molecular of 4,000-8,000.

Unless stated otherwise, all molecular weight values are g/mol, and all average molecular weight values are number-average molecular weight values.

According to the present invention, methyl polyglycolate or its product is first depolymerized to a depolymerized product. The depolymerized product may be a glycolate, a glycolate polymer, a glycolate prepolymer or a combination thereof. The depolymerized product may have a weight average molecular weight of less than 4,000. Depending on the extent of the depolymerization reaction, a glycolic acid monomer may be obtained if further depolymerization is accomplished.

The depolymerization may be carried out in the presence of a depolymerization agent. The depolymerization agent may comprise one or more hydroxyl groups. The depolymerization agent may comprise an alkyl alcohol, an alkyl alkyd or an alkyl alkanoate.

The alkyl alcohol contains one or more hydroxyl groups. The alkyl alcohol is preferably an alkyl alcohol having only one hydroxyl group. For example, the alkyl alcohol may be methanol.

The alkyl alkyd may comprise one or more hydroxyl groups and one or more carboxyl groups. In the alkyl alkyd, the number ratio of the hydroxyl groups to the carboxyl groups in the alkyl alkyd may be from 0.1:1 to 10:1. The number ratio is preferably 1:1. For example, the alkyl alkyd is glycolic acid.

The alkyl alkanoate may comprise one or more hydroxyl groups and one or more ester groups. In alkyl alkanoate, the number ratio of the hydroxyl group to the ester groups may be from 0.1:1 to 10:1. The number ration is preferably 1:1. For example, the alkyl alkanoate is methyl glycolate.

The alkyl alcohol, alkyl alkanoate, and alkyl alkyd added to the depolymerization reaction as depolymerization agents may remain in subsequent reactants and participate in the pyrolysis reaction to produce glycolide. These depolymerization agents do not affect the purity of the final glycolide product. At the same time, the preferred methanol, glycolic acid, and methyl glycolate are preferentially distilled out during the high temperature pyrolysis to generate glycolide due to their lower boiling point, and do not remain in the glycolide.

Therefore, the purity of the glycolide product is high. If the recovered methyl polyglycolate or its product is contaminated with sediment, the sediment can be simply washed and sieved with water.

Rare earth metal catalysts may be added to accelerate the depolymerization rate of high molecular weight methyl polyglycolate at lower temperatures during the depolymerization. The rare earth metal catalyst may be a metal oxide, a rare earth metal inorganic salt or a rare earth metal complex, each comprising lanthanum (La), cerium (Ce), praseodymium (Pr), neodynium (Nd), promethium (Pm), strontium (Sr) samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), scandium (Sc), or a combination thereof. The rare earth metal catalyst may comprise ruthenium, cobalt, rhodium, nickel or a combination thereof. The rare earth metal catalyst may be an oxide of ruthenium, cobalt, rhodium, nickel or a combination thereof. The rare earth metal catalyst may be present in an amount of about 0.0001-5 wt %, about 0.0006-4 wt %; more preferably about 0.001-2 wt %, based on the weight of the methyl polyglycolate or the methyl polyglycolate product.

The depolymerization reaction temperature may be about 25-260° C., preferably about 30-190° C., more preferably 40-120° C. The depolymerization reaction time may be about 0.5-48 h, preferably about 1-36 h, more preferably about 2-24 h. An increase in the reaction temperature promotes the depolymerization reaction.

The depolymerization reaction time may be about 0.5-48 h, preferably about 1-36 h, more preferably about 2-24 h. An increase in the reaction time promotes the depolymerization reaction.

The depolymerization may be carried out with microwave irradiation. At a relatively low heating temperature, intense microwave irradiation facilitates faster depolymerization, especially for methyl polyglycolate product having a high molecular weight. A temperature-controlled microwave equipment may be used to emit microwave irradiation for depolymerization. The emitted microwave output power may be about 1200-1600 W, for example, about 1400 W. The microwave is applied throughout the entire depolymerization process.

The depolymerized production is repolymerized to form a repolymerization mixture. The repolymerization mixture comprises a glycolic acid oligomer. The repolymerization reaction may be carried out about 100-300° C., 150-300° C. or 160-250° C. and under an absolute pressure of about 0.01-60.00 kPa or 0.05-50.00 kPa. The glycolic acid oligomer may have a weight average molecular weight of 4,000-80,000, and/or a free acid content of less than 2 wt %.

A glycolic acid oligomer having a weight average molecular weight in the range of 4,000 to 80,000 is desirable for producing glycolide with high purity and yield. The impurities and the depolymerization agent may be removed from the glycolic acid oligomer by distillation.

A repolymerization catalyst may be used to promote repolymerization. The repolymerization catalyst may be selected from the group consisting of zinc-based compounds, tin-based compounds, lanthanide-based compounds, and titanium-based compounds. A zinc-based, tin-based, antimony-based or titanium-based compound may be a compound comprising zinc, tin, antimony or titanium in any form, for example, a salt. Because the catalyst used for preparing the methyl polyglycolate and its product is not finally separated, the addition of a conventional polymerization catalyst such as a zinc-based, tin-based, antimony-based or titanium-based compound is no longer important in the repolymerization step.

A glycolic acid oligomer may be prepared by depolymerization and repolymerization. A glycolic acid oligomer having a lower free acid content can be obtained, thereby further reducing the free acid content to be 2% or less in the glycolide product prepared after pyrolysis. In addition, in the repolymerization reaction, impurities such as a depolymerization agent are separated by distillation so that high-purity glycolide can be obtained. The repolymerization process is not only advantageous for obtaining a glycolic acid oligomer having a weight average molecular weight of 4,000 to 80,000, but also advantageous for further purifying the intermediate product, thereby finally increasing the yield and purity of the glycolide.

In the pyrolysis reaction, repolymerization mixture is pyrolyzed and a glycolide product is obtained. The pyrolysis may be carried out at a temperature of about 200-300° C. or 230-280° C. under an absolute pressure from about 0.01-50 kPa or 0.05-30 kPa for 0.1-5 h or 0.5-2 h. No solvent may be added in the pyrolysis reaction.

A viscosity reducer may be added to facilitate the pyrolysis reaction. The viscosity reducer may have a boiling point greater than about 300, 330, 350 or 400° C. to enhance the melt flow. The viscosity reducer may be present in an amount of about 5-500 wt %, preferably about 10-100 wt %, more preferably about 15-60 wt %, based on the weight of the methyl polyglycolic acid or the methyl polyglycolate product. The viscosity reducer may be a polyether polyol, preferably a polyethylene glycol, polypropylene glycol or polybutylene glycol, more preferably a polyethylene glycol having a weight average molecular weight of about 1,500-20,000, most preferably a polyethylene glycol having a weight average molecular weight of about 1,500-8,000. The viscosity reducer may be a hydrocarbon mixture, preferably a mixture having a weight average molecular weight of less than about 25,000, more preferably a hydrocarbon mixture having a weight average molecular weight of about 1,000-15,000, most preferably about 1,500-8,000.

The viscosity reducer and the glycolide product may not be co-distilled. The pyrolysis reaction may comprise distilling the glycolide product without the viscosity reducer.

The pyrolysis reaction has a high pyrolysis conversion ratio. The term “pyrolysis conversion ratio” used herein refers to the percentage of glycolide product after a pyrolysis reaction over the total polyglycolate or a methyl polyglycolate product available at the beginning of the pyrolysis reaction. The pyrolysis conversion rate of this invention is greater than about 80%, 85%, 90% 95% or 99% of the glycolic acid oligomer.

For each process of the invention, a glycolide product prepared from the process is provided. The glycolide product is prepared from a methyl polyglycolate or a methyl polyglycolate product according to a process comprising depolymerizing the methyl polyglycolate or its product in the presence of a depolymerization agent to generate a depolymerized product, which may comprise glycolate, glycolate polymer or glycolate prepolymer; repolymerizing the depolymerized product to generate a repolymerized mixture comprising a glycolic acid oligomer; and pyrolyzing the repolymerized mixture. As a result, a glycolide product is prepared.

The glycolide product may have a free acid content of less than about 10, 5, 2 or 1 wt % based on the weight of the glycolide product.

A glycolic acid oligomer is also provided. The glycolic acid oligomer is prepared from a methyl polyglycolate or its product according to a process comprising depolymerizing the methyl polyglycolate or its product in the presence of a depolymerization agent to generate a depolymerized product, which may comprise glycolate, glycolate polymer or glycolate prepolymer; and repolymerizing the depolymerized product. As a result, a glycolic acid oligomer is prepared. The glycolic acid oligomer may have a weight average molecular weight of about 4,000-80,000. The glycolic acid oligomer may have a free acid content of less than about 10, 5, 2 or 1 wt % based on the weight of the glycolide product.

Several test methods may be used to characterize the glycolide production process and the products generated throughout the process according to the invention.

1. Weight Average Molecular Weight

A sample is dissolved in a solution of 5 mmol/L sodium trifluoroacetate in hexafluoroisopropanol to prepare a solution of 0.05-0.3 wt % (mass fraction). The solution is then filtered with a 0.4 μm pore size polytetrafluoroethylene filter. 20 μL of the filtered solution is added to the gel permeation chromatography (GPC) injector for determination of molecular weight of the sample. Five standard molecular weights of methyl methacrylate with different molecular weights are used for molecular weight correction.

2. Free Acid

0.5 g sample and about 20 ml of dimethyl sulfoxide are added into an Erlenmeyer flask to make sample solution. 0.01 mol/L potassium hydroxide solution is used to titrate the sample solution to detect the free acid of the sample.

3. Residue viscosity

The viscosity of a sample at 230° C. was measured using a rotational viscometer.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Depolymerization

### A. Depolymerized Products

28 depolymerized products were prepared as described below.

Depolymerization 1: 100 g of recovered methyl polyglycolate having a weight average molecular weight of 30,000 and 100 g of methanol were placed in an autoclave, heated to 180° C., reacted for 24 h, and cooled to normal temperature. A methyl glycolic acid oligomer was obtained from the filter cake after filtration, and had a weight average molecular weight of about 500. Methyl glycolate was detected in the filtrate. Thus, depolymerized product a was obtained.

Depolymerization 2: 100 g of recovered methyl polyglycolate having a weight average molecular weight of 30,000 and 100 g of methanol were placed in an autoclave, heated to 180° C., reacted for 12 h, and cooled to normal temperature. A methyl glycolic acid oligomer was obtained from the filter cake after filtration, and had a weight-average molecular weight of about 1,400. Methyl glycolate was detected in the filtrate. Thus, depolymerized product b was obtained.

Depolymerization 3: 100 g of recovered methyl polyglycolate having a weight average molecular weight of 30,000 and 100 g of ethanol were placed in an autoclave, heated to 180 ° C., reacted for 24 h, and cooled to normal temperature. A methyl glycolic acid oligomer was obtained from the filter cake after filtration, and had a weight-average molecular weight of about 300. Methyl glycolate was detected in the filtrate. Thus, depolymerized product c was obtained.

Depolymerization 4: 100 g of recovered methyl polyglycolate having a weight average molecular weight of 30,000 and 100 g of ethanol were placed in an autoclave, heated to 180° C., reacted for 24 h, and cooled to normal temperature. A methyl glycolic acid oligomer was obtained from the filter cake after filtration, and had a weight average molecular weight of about 450. Methyl glycolate was detected in the filtrate. Thus, depolymerized product d was obtained.

Depolymerization 5: 100 g of recovered methyl polyglycolate having a weight average molecular weight of 30,000 and 100 g of methyl glycolate were placed in an autoclave, heated to 180° C., reacted for 24 h, and cooled to normal temperature. A methyl glycolic acid oligomer was obtained from the filter cake after filtration, and had a weight average molecular weight of about 500. Methyl glycolate was detected in the filtrate. Thus, depolymerized product e was obtained.

Depolymerization 6: 50 g of recovered methyl polyglycolate having a weight average molecular weight of 30,000 and 50 g of recovered methyl polyglycolate having a weight average molecular weight of 80,000 were placed in an autoclave, heated to 180° C., reacted for 24 h, and cooled to normal temperature. A methyl glycolic acid oligomer was obtained from the filter cake after filtration, and had a weight average molecular weight of about 700. Methyl glycolate was detected in the filtrate. Thus, depolymerized product f was obtained.

Depolymerization 7: 40 g of recovered methyl polyglycolate having a weight average molecular weight of 3,000, 30 g of recovered methyl polyglycolate having a weight average molecular weight of 30,000, 30 g of recovered methyl polyglycolate having a weight average molecular weight of 80,000 and 100 g of methanol were placed in an autoclave, heated to 180° C., reacted for 24 h, and cooled to normal temperature. A methyl glycolic acid oligomer was obtained from the filter cake after filtration, and had a weight average molecular weight of about 500. Methyl glycolate was detected in the filtrate. Thus, depolymerized product g was obtained.

Depolymerization 8: 40 g of recovered methyl polyglycolate having a weight average molecular weight of 1,700, 30 g of recovered methyl polyglycolate having a weight average molecular weight of 80,000, 50 g of recovered methyl polyglycolate having a weight average molecular weight of 250,000 and 100 g of methanol were placed in an autoclave, heated to 180° C., reacted for 24 h, and cooled to normal temperature. A methyl glycolic acid oligomer was obtained from the filter cake after filtration, and had a weight average molecular weight of about 1,100. Methyl glycolate was detected in the filtrate. Thus, depolymerized product h was obtained.

Depolymerization 9: 100 g of recovered methyl polyglycolate having a weight average molecular weight of 30,000 and 100 g of methanol were placed in an autoclave, heated to 120° C., reacted for 24 h, and cooled to normal temperature. A methyl glycolic acid oligomer was obtained from the filter cake after filtration, and had a weight average molecular weight of about 780. Methyl glycolate was detected in the filtrate. Thus, depolymerized product i was obtained.

Depolymerization 10: 100 g of recovered methyl polyglycolate having a weight average molecular weight of 30,000 and 100 g of methanol were placed in an autoclave, heated to 120° C., reacted for 12 h, and cooled to normal temperature. A methyl glycolic acid oligomer was obtained from the filter cake after filtration, and had a weight average molecular weight of about 1200. Methyl glycolate was detected in the filtrate. Thus, depolymerized product j was obtained.

Depolymerization 11: 100 g of recovered methyl polyglycolate having a weight average molecular weight of 30,000 and 100 g of methanol were placed in an autoclave, heated to 120° C., reacted with simultaneously microwave irradiation for 12 h, and cooled to normal temperature. A methyl glycolic acid oligomer was obtained from the filter cake after filtration, and had a weight average molecular weight of about 300. Methyl glycolate was detected in the filtrate. Thus, depolymerized product k was obtained.

Depolymerization 12: 100 g of recovered methyl polyglycolate having a weight average molecular weight of 30,000 and 100 g of methanol were placed in an autoclave, heated to 120° C., reacted with simultaneously microwave irradiation for 8 h, and cooled to normal temperature. A methyl glycolic acid oligomer was obtained from the filter cake after filtration, and had a weight average molecular weight of about 450. Methyl glycolate was detected in the filtrate. Thus, depolymerized product I was obtained.

Depolymerization 13: 100 g of recovered methyl polyglycolate having a weight average molecular weight of 30,000, 100 g of methanol, and 0.01 g of La₂O₃ were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 190. Methyl glycolate was detected in the filtrate. Thus, depolymerized product m was obtained.

Depolymerization 14: 100 g of recovered methyl polyglycolate having a weight average molecular weight of 30,000, 100 g of methanol, and 0.01 g of Ce(HCO₃)₄ were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 160. Methyl glycolate was detected in the filtrate. Thus, depolymerized product n was obtained.

Depolymerization 15: 100 g of recovered methyl polyglycolate having a weight average molecular weight of 30,000, 100 g of methanol, and 0.01 g of lanthanum were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 210. Methyl glycolate was detected in the filtrate. Thus, depolymerized product o was obtained.

Depolymerization 16: 100 g of recovered methyl polyglycolate having a weight average molecular weight of 500,000 and 100 g of methanol were placed in an autoclave, heated to 120° C., reacted for 12 h, and cooled to normal temperature. A methyl glycolic acid oligomer was obtained from the filter cake after filtration, and had a weight average molecular weight of about 16,000. Methyl glycolate was detected in the filtrate. Thus, depolymerized product p was obtained.

Depolymerization 17: 100 g of recovered methyl polyglycolate having a weight average molecular weight of 500,000 and 100 g of methanol were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 6,000. Methyl glycolate was detected in the filtrate. Thus, depolymerized product q was obtained.

Depolymerization 18: 100 g of recovered methyl polyglycolate having a weight average molecular weight of 500,000 were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 1,200. Methyl glycolate was detected in the filtrate. Thus, depolymerized product r was obtained.

Depolymerization 19: 100 g of recovered methyl polyglycolate having a weight average molecular weight of 700,000 and 100 g of methanol were placed in an autoclave, heated to 120° C., reacted for 12 h, and cooled to normal temperature. A methyl glycolic acid oligomer was obtained from the filter cake after filtration, and had a weight average molecular weight of about 32,000. Methyl glycolate was detected in the filtrate. Thus, depolymerized product s was obtained.

Depolymerization 20: 100 g of recovered methyl polyglycolate having a weight average molecular weight of 700,000 and 100 g of methanol were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 11,000. Methyl glycolate was detected in the filtrate. Thus, depolymerized product t was obtained.

Depolymerization 21: 100 g of recovered methyl polyglycolate having a weight average molecular weight of 700,000, 100 g of methanol, and 0.01 g of Ce(HCO₃)₄ were placed in an autoclave, heated to 120° C., reacted with simultaneously microwave irradiation for 12 h, and cooled to normal temperature. A methyl glycolic acid oligomer was obtained from the filter cake after filtration, and had a weight average molecular weight of about 1,800. Methyl glycolate was detected in the filtrate. Thus, depolymerized product u was obtained.

Depolymerization 22: 100 g of recovered methyl polyglycolate having a weight average molecular weight of 900,000 and 100 g of methanol were placed in an autoclave, heated to 120° C., reacted for 12 h, and cooled to normal temperature. A methyl glycolic acid oligomer was obtained from the filter cake after filtration, and had a weight average molecular weight of about 43,000. Methyl glycolate was detected in the filtrate. Thus, depolymerized product v was obtained.

Depolymerization 23: 100 g of recovered methyl polyglycolate having a weight average molecular weight of 900,000 and 100 g of methanol were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 17,000. Methyl glycolate was detected in the filtrate. Thus, depolymerized product w was obtained.

Depolymerization 24: 100 g of recovered methyl polyglycolate having a weight average molecular weight of 900,000, 100 g of methanol, and 0.01 g of Ce(HCO₃)₄ were placed in an autoclave, heated to 120 ° C., reacted with simultaneously applied microwave irradiation for 12 h, and cooled to normal temperature. A methyl glycolic acid oligomer was obtained from the filter cake after filtration, and had a weight average molecular weight of about 1,900. Methyl glycolate was detected in the filtrate. Thus, depolymerized product x was obtained.

Depolymerization 25: 100 g of recovered methyl polyglycolate having a weight average molecular weight of 30,000, 100 g of methanol, and 0.01 g of Ce(HCO₃)₄ were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 190. Methyl glycolate was detected in the filtrate. Thus, depolymerized product y was obtained.

Depolymerization 26: 100 g of recovered methyl polyglycolate having a weight average molecular weight of 30,000, 100 g of methanol, and 0.0002 g of Ce(HCO₃)₄ were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 300. Methyl glycolate was detected in the filtrate. Thus, depolymerized product z was obtained.

Depolymerization 27: 100 g of recovered methyl polyglycolate having a weight average molecular weight of 30,000, 100 g of methanol, and 4 g of Ce(HCO₃)₄ were placed in an autoclave, heated to 120° C., reacted with simultaneously applied microwave irradiation for 12 h, and cooled to normal temperature. A methyl glycolic acid oligomer was obtained from the filter cake after filtration, and had a weight average molecular weight of about 180. Methyl glycolate was detected in the filtrate. Thus, depolymerized product 6 was obtained.

Depolymerization 28: 100 g of recovered methyl polyglycolate having a weight average molecular weight of 30,000, 100 g of methanol, and 4 g of Ce(HCO₃)₄ were placed in an autoclave, heated to 120° C., reacted for 12 h, and cooled to normal temperature. A methyl glycolic acid oligomer was obtained from the filter cake after filtration, and had a weight average molecular weight of about 820. Methyl glycolate was detected in the filtrate. Thus, depolymerized product β was obtained.

### B. Repolymerized Products

Nine depolymerized products were prepared as described below.

Repolymerization 1: 100 g of the depolymerized product a was gradually heated from 160° C. to 230° C. in an autoclave. The pressure was controlled at an absolute pressure of 3 kPa. The methanol formed during the reaction was continuously removed for 2 h. The resulting glycolic acid oligomer A had a weight average molecular weight of 6,200 and a free acid content of 1.3 wt %.

Repolymerization 2: 100 g of the depolymerized product a was gradually heated from 160° C. to 230° C. in an autoclave. The pressure was controlled at an absolute pressure of 3 kPa. The methanol formed during the reaction was continuously removed for 6 h. The resulting glycolic acid oligomer B had a weight average molecular weight of 8,200 and a free acid content of 0.9 wt %.

Repolymerization 3: 100 g of the depolymerized product a was gradually heated from 160° C. to 230° C. in an autoclave. The pressure was controlled at an absolute pressure of 3 kPa. The methanol formed during the reaction was continuously removed for 8 h. The resulting glycolic acid oligomer C had a weight average molecular weight of 15,000 and a free acid content of 0.7 wt %.

Repolymerization 4: 100 g of the depolymerized product a and 0.5 g of stannous octoate were gradually heated from 160° C. to 230° C. in an autoclave. The pressure was controlled at an absolute pressure of 3 kPa. The methanol formed during the reaction was continuously removed for 6 h. The resulting glycolic acid oligomer D had a weight average molecular weight of 9,400 and a free acid content of 0.6 wt %.

Repolymerization 5: 100 g of the depolymerized product a was gradually heated from 160° C. to 230° C. in an autoclave. The pressure was controlled at an absolute pressure of 50 kPa. The methanol formed during the reaction was continuously removed for 8 h. The resulting glycolic acid oligomer E had a weight average molecular weight of 5,500 and a free acid content of 0.7 wt %.

Repolymerization 6: 100 g of the depolymerized product d was gradually heated from 160° C. to 230° C. in an autoclave. The pressure was controlled at an absolute pressure of 3 kPa. The methanol formed during the reaction was continuously removed for 6 h. The resulting glycolic acid oligomer F had a weight average molecular weight of 7,900 and a free acid content of 0.9 wt %.

Repolymerization 7: 100 g of the depolymerized product e was gradually heated from 160° C. to 230° C. in an autoclave. The pressure was controlled at an absolute pressure of 3 kPa. The methanol formed during the reaction was continuously removed for 6 h. The resulting glycolic acid oligomer G had a weight average molecular weight of 7,900 and a free acid content of 0.8 wt %.

Repolymerization 8: 100 g of the depolymerized product e was gradually heated from 160° C. to 230° C. in an autoclave. The pressure was controlled at an absolute pressure of 70 kPa. The methanol formed during the reaction was continuously removed for 6 h. The resulting glycolic acid oligomer H had a weight average molecular weight of 43,000 and a free acid content of 1.1 wt %.

Repolymerization 9: 100 g of the depolymerized product e was gradually heated from 160° C. to 230° C. in an autoclave. The pressure was controlled at an absolute pressure of 70 kPa. The methanol formed during the reaction was continuously removed for 8 h. The resulting glycolic acid oligomer I had a weight average molecular weight of 72,000 and a free acid content of 1.3 wt %.

C. Pyrolysis products

Five pyrolysis products were prepared as described below.

Pyrolysis 1: 100 g of the glycolic acid oligomer A was subjected to a pyrolysis reaction at 250° C. under an absolute pressure of 1 kPa for 2 hours, and distilled to a glycolide product. The glycolide product was collected by condensing it with ice water. The glycolide pyrolysis yield was 90%. The free acid content was 1.9 wt %. The rotational viscosity of the pyrolysis residue at 230° C. was 0.35 Pa·s.

Pyrolysis 2:100 g of the glycolic acid oligomer B was subjected to a pyrolysis reaction at 250° C. under an absolute pressure of 1 kPa for 2 hours, and distilled to a glycolide product. The glycolide product was collected by condensing it with ice water. The glycolide pyrolysis yield was 91%. The free acid content was 1.5%. The rotational viscosity of the pyrolysis residue at 230° C. was 0.67 Pa·s.

Pyrolysis 3: 100 g of the glycolic acid oligomer C was subjected to a pyrolysis reaction at 250° C. under an absolute pressure of 1 kPa for 2 hours, and distilled to a glycolide product. The glycolide product was collected by condensing it with ice water. The glycolide pyrolysis yield was 91%. The free acid content was 1.1 wt %. The rotational viscosity of the pyrolysis residue at 230° C. was 0.72 Pa·s.

Pyrolysis 4: 100 g of the glycolic acid oligomer C and 40 g of polyethylene glycol (molecular weight 4000) were subjected to a pyrolysis reaction at 250° C. under an absolute pressure of 1 kPa for 2 hours, and distilled to a glycolide product. The glycolide product was collected by condensing it with ice water. The glycolide pyrolysis yield was 94%. The free acid content was 0.9 wt %. The rotational viscosity of the pyrolysis residue at 230° C. was 0.13 Pa·s.

Pyrolysis 5: 100 g of the glycolic acid oligomer C and 40 g of paraffin wax (molecular weight 1500) were subjected to a pyrolysis reaction at 250° C. under an absolute pressure of 1 kPa for 2 hours, and distilled to a glycolide product. The glycolide product was collected by condensing it with ice water. The glycolide pyrolysis yield was 95%. The free acid content was 1.0 wt %. The rotational viscosity of the pyrolysis residue at 230° C. was 0.22 Pa·s.

D. Comparative products

Two comparative products were prepared as described below.

Comparative 1: 100 g of recovered methyl polyglycolate having a weight average molecular weight of 10,000 and a free acid content of 11.4 wt % was subjected to a pyrolysis reaction at 250° C. under an absolute pressure of 1 kPa for 2 hours, and distilled to a glycolide product. The glycolide product was collected by condensing it with ice water. The glycolide pyrolysis yield was 69%. The free acid content was 15.2 wt %. The rotational viscosity of the pyrolysis residue at 230° C. was 1.03 Pa·s.

Comparative 2: 100 g of recovered methyl polyglycolate having a weight average molecular weight of 180,000 and a free acid content of 0.9 wt % was subjected to a pyrolysis reaction at 250° C. under an absolute pressure of 1 kPa for 2 hours, and distilled to a glycolide product. The glycolide product was collected by condensing it with ice water. The glycolide pyrolysis yield was 63%. The free acid content was 2.2 wt %. The rotational viscosity of the pyrolysis residue at 230° C. was 13.54 Pa·s.

Although the invention is illustrated and described herein with reference to specific embodiments, the invention is not intended to be limited to the details shown. Rather, various modifications may be made in the details within the scope and range of equivalents of the claims without departing from the invention.

1. A process of preparing a glycolide product from a methyl polyglycolate or a methyl polyglycolate product, comprising:
(a) depolymerizing a methyl polyglycolate or methyl polyglycolate product in the presence of a depolymerization agent, whereby a depolymerized product is generated, wherein the depolymerized product comprises glycolate, glycolate polymer or glycolate prepolymer;
(b) repolymerizing the depolymerized product, whereby a repolymerized mixture is generated, wherein the repolymerized mixture comprises a glycolic acid oligomer; and
(c) pyrolyzing the repolymerized mixture, whereby a glycolide product is prepared.

2. The process of claim 1, wherein the depolymerization agent comprises one or more hydroxyl groups and comprises an alkyl alcohol, an alkyl alkyd or an alkyl alkanoate.

3. The process of claim 2, wherein the alkyl alcohol comprises one hydroxyl group.

4. The process of claim 2, wherein the alkyl alkyd comprises one or more hydroxyl groups and one or more carboxyl groups at a number ratio of the hydroxyl groups to the carboxyl groups in a range from 0.1:1 to 10:1.

5. The process of claim 2, wherein the alkyl alkanoate comprises one or more hydroxyl groups and one or more ester groups at a number ratio of the hydroxyl groups to the ester groups in a range from 0.1:1 to 10:1.

6. The process of claim 1, wherein step (a) is carried out at 25-260° C. for 0.5-48 h.

7. The process of claim 1, further comprising applying microwave irradiation to control temperature in step (a), whereby depolymerization is accelerated.

8. The process of claim 1, wherein step (a) is carried out in the presence of a rare earth metal catalyst, wherein the rare earth metal catalyst is a metal oxide, a rare earth metal inorganic salt or a rare earth metal complex comprising lanthanum (La), cerium (Ce), praseodymium (Pr), neodynium (Nd), promethium (Pm), strontium (Sr), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), scandium (Sc), or a combination thereof.

9. The process of claim 8, wherein the rare earth metal catalyst is present in an amount of 0.0001-5 wt %, based on the weight of the methyl polyglycolate or the methyl polyglycolate product.

10. The process of claim 1, wherein the glycolic acid oligomer has a weight average molecular weight of 4,000-80,000 and a free acid content of less than 2 wt %.

11. The process of claim 1, wherein step (b) is carried out in the presence of a repolymerization catalyst selected from the group consisting of zinc-based compounds, tin-based compounds, lanthanide-based compounds, and titanium-based compounds.

12. The process of claim 1, wherein step (b) is carried out at 160-250° C. and under an absolute pressure of 0.05-50.00 kPa.

13. The process of claim 1, wherein step (c) is carried out at 230-280° C. and an absolute pressure of 0.05-30.00 kPa.

14. The process of claim 1, wherein step (c) is carried out in the presence of a viscosity reducer having a boiling point greater than 330° C., and further comprising distilling the glycolide product without the viscosity reducer.

15. The process of claim 14, wherein the viscosity reducer is present in an amount of 5-500 wt % based on the weight of the methyl polyglycolic acid or the methyl polyglycolate product.

16. The process of claim 14, wherein the viscosity reducer is a polyether polyol.

17. The process of claim 14, wherein the viscosity reducer is a hydrocarbon mixture.

18. The process of claim 1, wherein the methyl polyglycolate or the methyl polyglycolate product has a weight average molecular weight of 1,000-1,000,000.

19. The process of claim 1, wherein step (c) has a pyrolysis conversion ratio of more than 90% of the glycolic acid oligomer and generates an incompletely converted glycolid acid oligomer.

20. The process of claim 19, further comprising discharging the incompletely converted glycolic acid oligomer as a residue at 230° C. with a dynamic viscosity less than 1 Pa·s.

21. A glycolide product prepared from a methyl polyglycolate or a methyl polyglycolate product according to a process, wherein the process comprises:
(a) depolymerizing a methyl polyglycolate or methyl polyglycolate product in the presence of a depolymerization agent, whereby a depolymerized product is generated, wherein the depolymerized product comprises glycolate, glycolate polymer or glycolate prepolymer;
(b) repolymerizing the depolymerized product, whereby a repolymerized mixture is generated, wherein the repolymerized mixture comprises a glycolic acid oligomer; and
(c) pyrolyzing the repolymerized mixture, whereby a glycolide product is prepared.

22. The glycolide product of claim 21, wherein the glycolide product has a free acid content of less than 2%.

23. A glycolic acid oligomer prepared from a methyl polyglycolate or a methyl polyglycolate product according to a process, wherein the process comprises:
(a) depolymerizing a methyl polyglycolate or methyl polyglycolate product in the presence of a depolymerization agent, whereby a depolymerized product is generated, wherein the depolymerized product comprises glycolate, glycolate polymer or glycolate prepolymer; and
(b) repolymerizing the depolymerized product, whereby a glycolic acid oligomer is prepared.

24. The glycolic acid oligomer of claim 23, wherein the glycolic acid oligomer has a weight average molecular weight of 4,000-80,000.

25. The glycolic acid oligomer of claim 23, wherein the glycolic acid oligomer has a free acid content of less than 2 wt %.
